(19) United States
(12) Patent Application Publication
(10) Pub. No.: US 2016/0193469 A1
(43) Pub. Date: Jul. 7, 2016

(54) NEUROMODULATION SYSTEM AND RELATED METHODS

(71) Applicant: Urologix, LLC, Minneapolis, MN (US)

(72) Inventors: Ralph Peter CARDINAL, White Bear Lake, MN (US); Gregory James FLUET, Minneapolis, MN (US)

(21) Appl. No.: 15/072,989

(22) Filed: Mar. 17, 2016

Related U.S. Application Data
(63) Continuation of application No. 13/851,760, filed on Mar. 27, 2013, now abandoned.
(60) Provisional application No. 61/616,324, filed on Mar. 27, 2012.

(51) Int. Cl. A61N 1/36; A61N 1/05; A61B 18/18

(52) U.S. Cl. CPC A61N 1/36021; A61B 18/1815; A61N 1/36007; A61N 1/0551; A61B 2018/00547

(57) ABSTRACT

Embodiments of the present invention describe a system and method of stimulating nerves during a medical procedure including positioning one or more electrodes in electrical contact with one or more nerves of a patient and electrically stimulating the one or more nerves during a lower urinary, rectal or medical procedure affecting the bowel sufficient to affect one or more of bladder activity, prostate function, incontinence, or pain.

### CROSS REFERENCE TO RELATED APPLICATIONS

This application is a continuation of U.S. application Ser. No. 13/851,760, filed on Mar. 27, 2013, which claims the benefit of U.S. Provisional Application No. 61/616,324, filed on 27 Mar. 2012, the entire contents of which are incorporated herein by reference.

### BACKGROUND

The prostate gland is a complex, chestnut-shaped organ which encircles the urethra immediately below the bladder and lies immediately adjacent the rectum. This relatively small organ, which is the most frequently diseased of all internal organs, is the site of a common affliction among older men, benign prostatic hyperplasia (BPH), as well as a more serious affliction, cancer. BPH is a non-malignant, nodular tumorous expansion of prostate tissue occurring mainly in the transition zone of the prostate. Left untreated, BPH causes obstruction of the urethra which usually results in increased urinary frequency, urgency, incontinence, nocturia and slow or interrupted urinary stream. BPH may also result in more severe complications, such as bladder decompensation, urinary tract infection, acute urinary retention, hydronephrosis and uraemia.

A common treatment method for BPH involves microwave thermal therapy, in which microwave energy is employed to elevate the temperature of tissue surrounding the prostatic urethra above about 45° C., thereby thermally damaging the tumorous BPH tissue. Delivery of microwave energy to tumorous prostatic tissue is generally accomplished by a microwave antenna-containing applicator, which is positioned within a body cavity adjacent the prostate gland. The microwave antenna, when energized, heats adjacent tissue due to molecular excitation and generates a radiation pattern which encompasses and necroses the tumorous prostatic tissue. The necrosed intraprostatic tissue is subsequently reabsorbed by the body, thereby relieving an individual from the symptoms of BPH.

One type of thermal therapy treatment of BPH is transurethral microwave thermal therapy. This method of treatment positions a Foley-type catheter containing a microwave antenna within the urethra adjacent to the prostate gland. The microwave antenna is energized to heat and necrose a selected volume of tumorous prostatic tissue up to 2.0 centimeters from the urethra, by raising the temperature of the selected tissue to a temperature above about 45° C. for a time sufficient to necrose the tissue.

Due to the relatively close proximity of the rectum to the urethra, it is critically important in the course of transurethral thermal therapy that the temperature of the rectum is maintained below a threshold temperature. Rectal temperatures greater than the threshold may cause significant damage to the rectum.

The variety of urinary symptoms associated with BPH is generally referred to as LUTS (Lower Urinary Tract Syndromes). While all urinary symptoms caused by BPH fall under the LUTS designation, not all LUTS is caused by BPH. Over Active Bladder (OAB) is a condition when the bladder has frequent spasms that result in urgency and patient discomfort. Symptoms from OAB also fall under the designation of LUTS. Differential diagnosis of LUTS related to BPH and LUTS related to OAB can be challenging. Theoretically, BPH and OAB may be related as OAB may result from increased stresses on bladder function from BPH and the resultant Bladder Outlet Obstruction (BOO) that it may cause. Historically when diagnosis of LUTS related to BPH is made the Prostate is targeted for therapy. However, it is getting more common to find patients diagnosed with BPH who are also being treated pharmacologically for OAB as well.

One of the primary symptoms of OAB is a sense of urinary urgency with the bladder spasms, even if there is no need to void. Bladder spasms resulting in urgency is a common source of discomfort during TUMT procedures. It may be that the patient has OAB which is causing these spasms. For patients without OAB, theoretically there may be some neural response to the microwave energy that is causing the bladder spasms, or more likely the presence of the catheter and distal balloon on the bladder sphincter may cause the bladder spasms temporarily during the procedure.

Optimal management of patient discomfort is a common objective for many in-office urological procedures where general anesthesia is not used. These include TUMT, TUNA, prostate biopsy, cystoscopy, etc. Several methods have been employed to mitigate the discomfort and pain; including transrectal prostatic blocks using lidocaine, transurethral lidocaine injections, and ice packs in the abdominal area as well as oral medications to sedate and comfort the patient. Bladder spasms, whether related to OAB or due to spasms during a TUMT procedure are often controlled with drugs that are anti-spasmodics or anti-cholinergics.

### SUMMARY

Embodiments of the present invention describe a system and method of stimulating nerves during a medical procedure including positioning one or more electrodes in electrical contact with one or more nerves of a patient and electrically stimulating the one or more nerves during a lower urinary, rectal or medical procedure affecting the bowel sufficient to affect one or more of bladder activity, prostate function, incontinence, or pain.

### BRIEF DESCRIPTION OF DRAWINGS

FIG. 1 shows a block flow diagram of a method of stimulating nerves during a medical procedure, according to some embodiments.

FIG. 2 shows a schematic diagram of a neuromodulation system, according to some embodiments.

FIG. 3 shows a vertical sectional view of a male pelvic region illustrating a transurethral thermal therapy device positioned in the urethra and a rectal thermosensing unit (RTU) positioned within the rectum of the male pelvic region, according to some embodiments.

FIG. 4 shows a perspective view of a rectal thermosensing unit, according to some embodiments.

FIG. 5 shows a schematic view of a nerve stimulator in use on a patient, according to some embodiments.

FIG. 6 shows a perspective view of a nerve stimulator in use on a patient, according to some embodiments.

FIG. 7 shows a perspective view of a pelvic nerve system, according to embodiments.

FIG. 8 shows a schematic view of a nerve stimulator system, according to some embodiments.

FIG. 9 shows a perspective view of electrode configurations of a nerve stimulator system, according to some embodiments.

### DETAILED DESCRIPTION

Embodiments of the present invention disclose a system and methods for treating patient discomfort or pain during a lower urinary, prostatic or rectal medical procedures. Such a procedure may be a transurethral energy based procedure. Beyond managing discomfort in BPH related procedures, the embodiments can be used as an electrical pain block for prostate biopsies or other prostatic therapies by inhibiting prostatic and rectal nociception. Electrical stimulation of one or more nerves controls the function or reaction of the bladder or other organ that affects patient discomfort or pain. The electrodes can be placed in one or more locations, including integrated with current BPH devices and other stimulation or monitoring devices which are also used to modulate bladder spasms when deployed to solely treat OAB related spasms. These are devices such as Medtronic's Interstim device or Uroplasty's percutaneous tibial nerve stimulation (PTNS) device.

Reviewing neuroanatomy, it can be concluded that blocking, exciting, inhibiting or otherwise stimulating (i.e., electrically contacting) the nerves that innervate the prostatic region or the bladder would help to relieve the discomfort or pain that can be associated with in-office prostatic therapies. In addition, it may help to alleviate additional symptomology of LUTS related to OAB, not solely caused by BPH if nerves within the sacral nerve plexus pathway are targeted.

A method of electrically blocking, exciting or inhibiting afferent and efferent nerve fibers that innervate the prostatic tissue using an array of electrodes or a single electrode is proposed. The electrodes could be placed into the rectum in an embodiment, but could also be placed transurethrally, in the bladder, transperineally, or even remote to the treatment site. Remote placement could include stimulating peripheral nerves along the targeted neural pathways such as the posterior tibial nerve or positions in the spine. In addition, the therapy could be delivered through implantable neural stimulators known in the art. As an example: using the current Urologix® CTT system with modifications to the rectal thermal sensing unit (RTU); one or more electrodes could be delivered in conjunction with the therapy and delivery of the RTU.

In addition, peripheral nerves could be stimulated intra-procedurally which connect to these same neural pathways to remotely stimulate, block, excite or inhibit the nerves to control the discomfort caused by a sense of urgency related to bladder spasms.

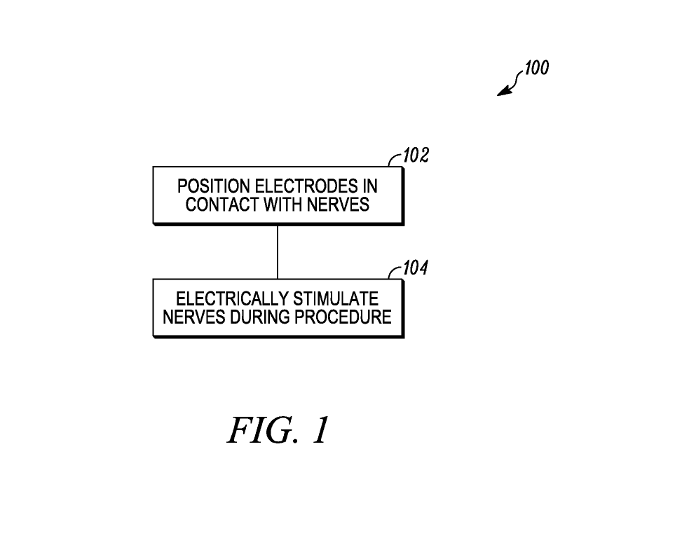

Referring to FIG. 1, a block flow diagram 100 of a method of stimulating nerves during a medical procedure is shown, according to embodiments of the present invention. One or more electrodes are positioned 102 in electrical contact with one or more nerves within or in proximity to a rectum of a patient. The one or more nerves are electrically stimulated 104 during a lower urinary or bowel medical procedure sufficient to affect one or more of bladder activity, prostate function, incontinence and pain.

One or more of the electrodes can optionally be in electrical contact with one or more nerves in one or more of the bladder, perineum, urethra and leg. For example, electrodes can be simultaneously placed transurethrally as part of a BPH treatment or device and also in the rectum as part of a rectal thermosensing unit. In another example, electrodes can be placed in the urethra while treating a urinary condition and also in the foot using a PTNS device. If electrodes are placed in more than one location, they can be stimulated simultaneously or sequentially and any portion of before, during and after a medical procedure.

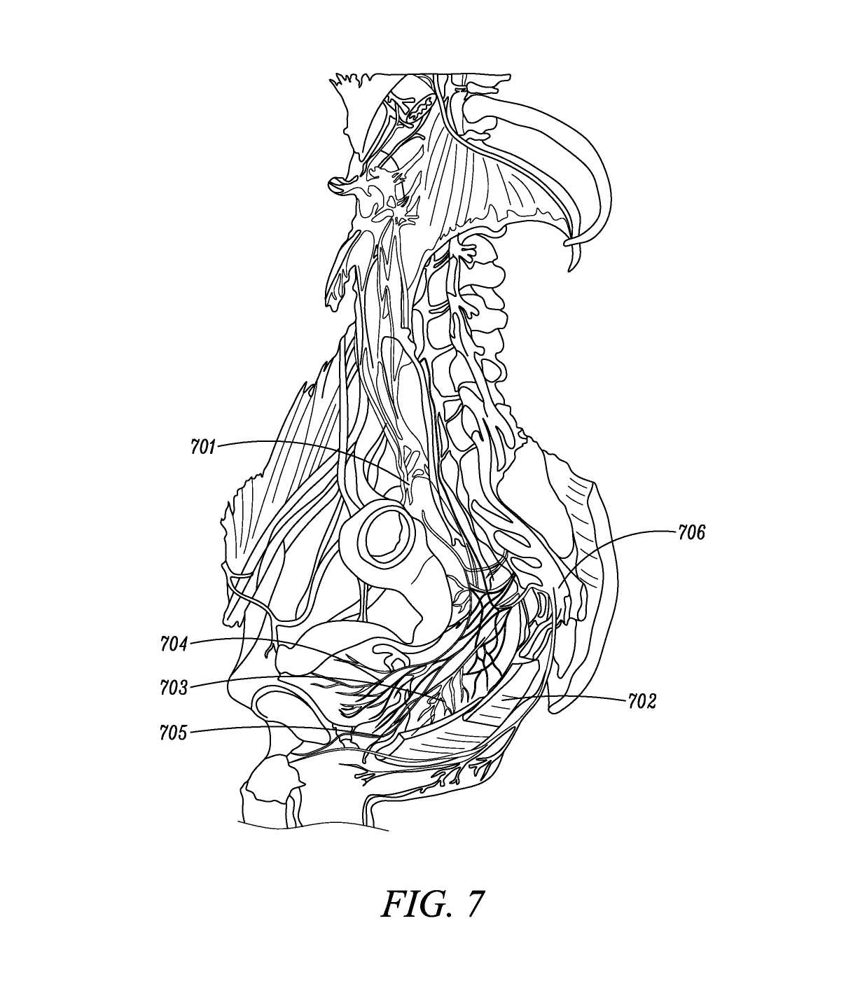

Examples of the one or more nerves are shown in FIG. 7, include nerves of the pelvis, hypogastric (splanchnic) 701, perineal and pudendal nerves 702. Further examples include rectal 703, vesical 704 and prostatic nerve plexuses 705. Sacral nerves 706 may also be stimulated, excited, inhibited or blocked, for example. Specific nerves or groups of nerves to be targeted depend on the type of treatment desired. For example, the perineal nerve is often associated with lower urinary tract procedures and therapies

Examples of medical procedure include one or more of transurethral needle ablation of the prostate (TUNA), transurethral microwave thermotherapy of the prostate (TUMT), prostate biopsy, colon polyp removal and cystoscopy. For procedures involving the bowel such as colon polyp removal, stimulation could be used to block pain resulting from polyp excision or similar procedures. The one or more electrodes can be a single electrode or an array of electrodes, as an example. The electrodes can be integrated into existing medical devices, such as microwave catheters, PTNS devices, thermosensing units or probes. In the rectum, for example, the electrodes can be arranged in one or more rings, rows or other arrangement on the outer surface of a balloon probe or catheter.

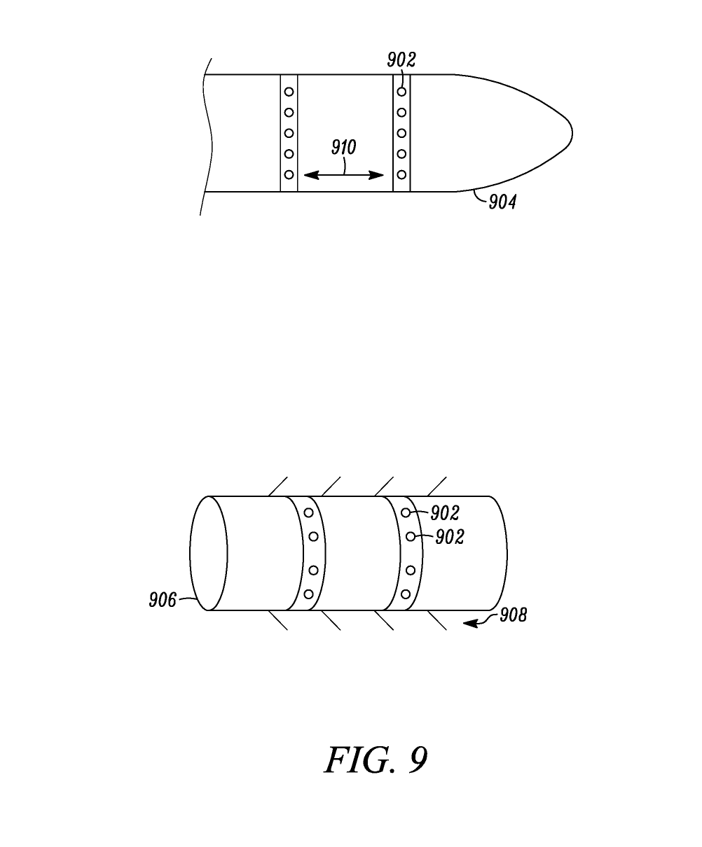

For example, FIG. 9 shows possible electrode 902 configurations on an RTU balloon 904, rectal catheter or sensing unit 906 or both. The one or more electrodes 902 can include one or more bands, positioned circumferentially around one or more sections of the balloon 904 or catheter 906. Such sections can include a distal region, proximal region or sections positioned between a proximal or distal region of the balloon 904 or catheter 906. The one or more electrodes 902 may be positioned partially circumferentially, at a distal tip, in a staggered formation, in bands or rows parallel to the length of the balloon 904 or catheter 906 or any configuration that provides for sufficient contact with the desired nerves and sensing capabilities. The spacing of the electrodes may be narrow if focused nerve stimulation is desired or wide if larger groups of nerves or a nerve plexus is being stimulated. In addition, if large areas need to be stimulated for a specific treatment or therapy, electrodes from one device such as the RTU balloon can work in combination with electrodes from a second device such as a catheter placed in the urethra. Further, a plurality of barbs 908 or other attachment or contacting mechanism can be utilized to hold the sensing device and electrodes in place.

Electrically stimulating includes providing sinusoidal waveforms, biphasic waveforms or both, with frequencies of about 1 kHz to about 100 kHz, about 5 kHz to about 75 kHz or about 15 kHz to about 50 kHz. The waveform currents can range from about 1 mA to about 10 mA, about 3 mA to about 12 mA or about 0.5 mA to about 15 mA, for example.

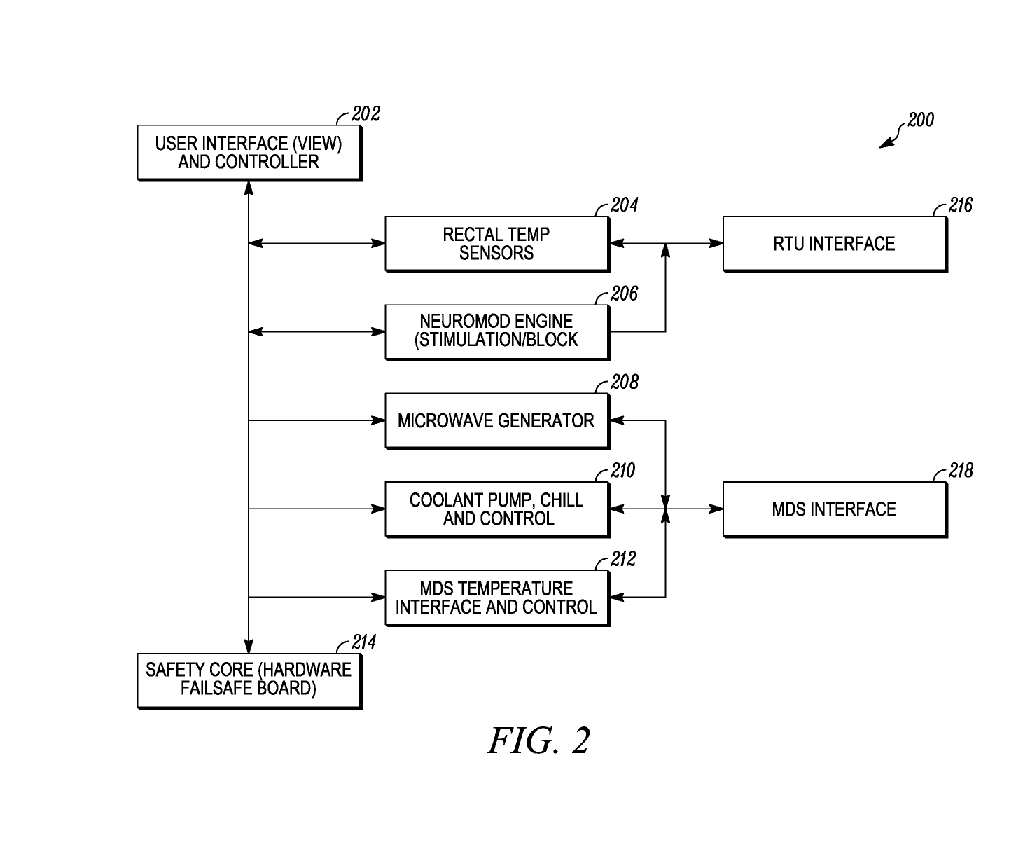

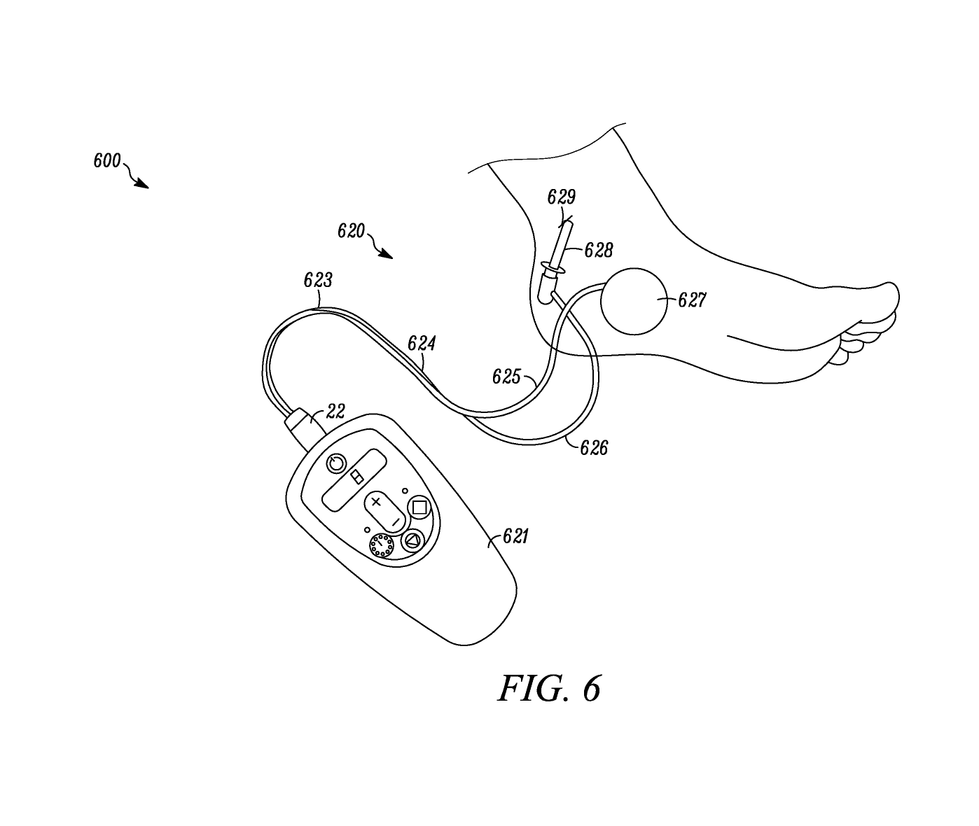

Referring to FIG. 2, a schematic diagram 200 of a neuromodulation system is shown, according to embodiments of the present invention. The system includes one or more electrodes 212, one or more temperature sensors 204, a microwave generator 208, an electrical stimulation module 206 in electrical communication with the one or more electrodes and a controller 202, in electrical communication with one or more of the one or more electrodes, one or more temperature sensors, microwave generator, and electrical stimulation module. The electrical stimulation module 206 can be a stand-alone device as shown in FIG. 6 or part of another device providing therapy to the lower urinary track or bowl, such as a control unit for cooled thermal therapy. The one or more electrodes are capable of stimulating one or more nerves during a lower urinary or bowel medical procedure sufficient to affect one or more of bladder activity, prostate function, and incontinence. The system optionally includes a user interface view for the controller 202 and a coolant system 210. A safety core module 214, including a hardware failsafe board in conjunction with other software safety features, can be optionally in electrical contact with the controller 202.

The one or more temperature sensors include one or more of rectal temperature sensors and microwave delivery system (MDS) temperature sensors. Each type of temperature sensor can be associated with optional user interfaces 216, 218 respectively. The coolant module 210 includes coolant pump, coolant and coolant control module.

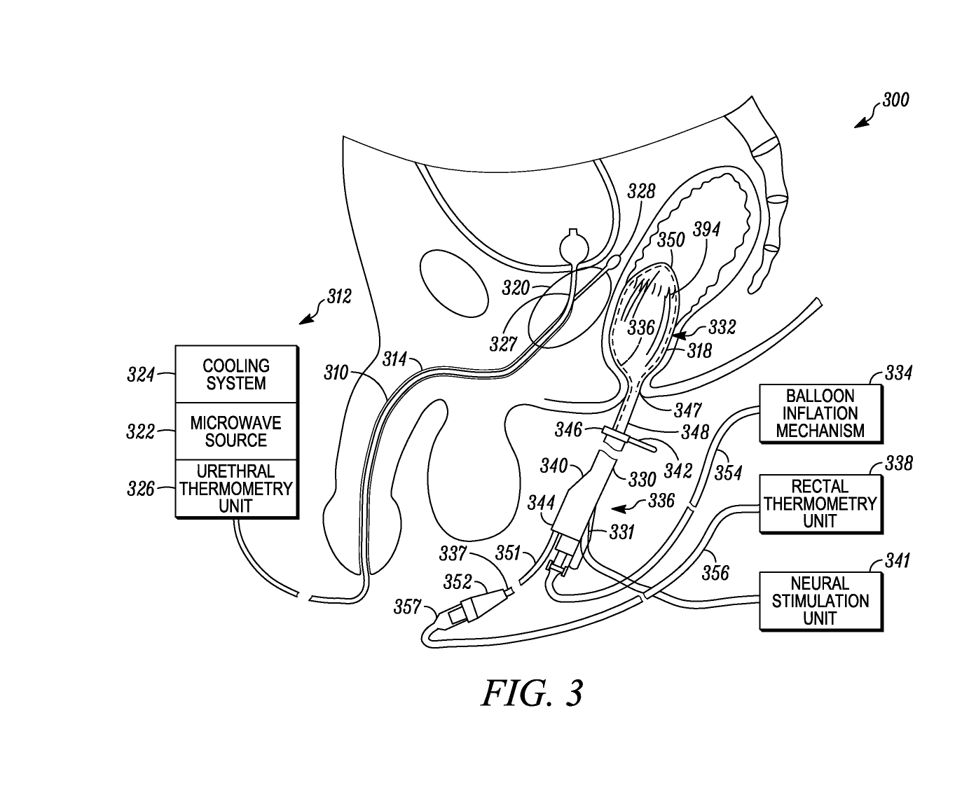

FIG. 3 shows a vertical sectional view 300 of a male pelvic region illustrating catheter 310 of transurethral thermal therapy system 312 properly positioned within urethra 314 and rectal thermosensing unit 316 properly positioned within rectum 318. The one or more neurostimulation electrodes 394 of the present embodiments can be integrated into the thermosensing unit 316, transurethral thermal therapy system or both, for example.

Transurethral thermal therapy system 312 heats benign tumorous tissue growth within prostate 320 surrounding urethra 314 to necrose the tumorous tissue. Catheter 310 of transurethral thermal therapy system 312 preferably comprises a microwave antenna-containing catheter including a multi-lumen shaft. Transurethral thermal therapy system 312 further includes a microwave source 322, a cooling system 324 and a urethral thermometry unit 326. As described in further detail in U.S. Pat. No. 5,413,588 entitled DEVICE FOR ASYMMETRICAL THERMAL THERAPY WITH HELICAL DIPOLE MICROWAVE ANTENNA, which is hereby incorporated by reference, transurethral thermal therapy system 312 treats benign tumorous tissue growth within prostate 320 with a microwave generating source 322, which energizes an antenna 327 located within catheter 310 and positioned within urethra 314 across prostate 320. Energization of antenna 327 causes antenna 327 to emit electromagnetic energy which heats tissue within prostate 320. To avoid unnecessary and undesirous damage to urethra 310 and adjacent healthy tissues, cooling system 324 supplies a cooling fluid through the multi-lumen shaft of catheter 310 to precisely control temperature distribution of tissue surrounding catheter 310 based upon temperatures of the tissues sensed by urethral thermometry unit 326.

To further measure and monitor the temperature of tissue adjacent prostate 320 so as to prevent unnecessary damage to rectum 318 and otherwise healthy tissue surrounding prostate 320, rectal thermosensing unit 316 is positioned within rectum 318 adjacent prostate 320. Rectal thermosensing unit 316 generally includes handle 330, control valve 331, balloon 332, balloon inflation mechanism 334, at least one temperature sensing device 336, neural stimulation unit 341, sensing device connector assembly 337 and rectal thermometry unit 338. Handle 330 is a generally elongate member having a central body 340 and a flag 342. Central body 340 includes a first end 344 and a second end 346. First end 344 of central body 340 is located adjacent control valve 331 and connector assembly 337. Second end 346 of handle 330 is coupled to balloon 332 and temperature sensing device 336. Central body 340 preferably has a length extending between first end 344 and second end 346 sufficient to allow a physician to easily grasp handle 330. Handle 330 preferably has a length of about 6.5 inches and a diameter of about 0.5 inches. Handle 330 enables a physician to easily manipulate balloon 332 and temperature sensing device 336 for properly positioning temperature sensing device 336 within rectum 318 adjacent prostate 320.

Flag 342 generally comprises an elongate protrusion radially extending outward from central body 340 at a selected angle or position relative to balloon 332 and temperature sensing device 336. Flag 342 is located at the second end 346 of handle 330 and indicates the orientation of balloon 318 and temperature sensing device 336 within rectum 318. Flag 342 further indicates when balloon 332 and temperature sensing device 336 have been fully inserted into rectum 318. Positioning can alternately be accomplished with an additional handle that engages with the catheter to ensure proper orientation. As can be appreciated, a variety of indicating mechanisms such as indexing marks, grooves or alternative projections may be used in lieu of flag 342 for permitting a physician to visually determine the orientation and location of balloon 332 and temperature sensing device 336 within rectum 318.

Control valve 331 preferably comprises a standard stop cock or one-way valve for regulating inflation of balloon 332 by balloon inflation mechanism 334. Control valve 331 is attached to handle 330 and is fluidly coupled to an inflation lumen 354 extending through handle 330. Control valve 331 regulates the flow of inflation fluid from balloon inflation mechanism 334 through inflation lumen 354 into balloon 332. Because control valve 331 is positioned adjacent handle 330, a physician may easily manipulate balloon 332 while also adjusting the rate of inflation of balloon 332.

Balloon 332, upon inflation, is a generally oval-shaped balloon having a first end 348 and a second end 350. First end 348 of balloon 332 is coupled to second end 346 of handle 330. In a preferred embodiment, balloon 332 is manufactured from a flexible, elastic material such as silicone or urethane. The rectal probe consisting of balloon 332 and its associated temperature sensing device 336 has a relatively low overall weight and is able to maintain its positioning within the rectum when inflated. Because balloon 332 is oval-shaped, balloon 332 has an extremely large surface area which may be positioned in contact with the rectal tissue upon inflation. Consequently, balloon 332, upon inflation, sufficiently engages the wall of the rectum to maintain balloon 332 and temperature sensing device 336 within the rectum during treatment without severely compressing rectal tissue. By avoiding compression of rectal tissue and the blood vessels therein, the risk of thermal damage to rectal tissue is substantially reduced.

Prior to a transurethral thermal therapy treatment, balloon 332 is inserted into rectum 318 in an uninflated state. Once inserted into rectum 318, balloon 332 is inflated by balloon inflation mechanism 334. Balloon inflation mechanism 334 preferably inflates balloon 332 with a gas such as air to a selected inflation volume and pressure so as to cause balloon 332 to assume its oval shape as closely as possible and to engage the rectal wall without compressing adjacent tissue. Because balloon 332 is preferably inflated with a gas such as air, rather than a liquid, the temperature effects of the inflation medium upon the temperature sensing device 336 are minimized, resulting in more accurate temperature measurements. In addition, because balloon 332 is preferably inflated with a gas, rather than a liquid, balloon 332 is lighter in weight and better maintains its positioning within the rectum. Upon being inflated by balloon inflation mechanism 334, balloon 332 positions and maintains temperature sensing device 36 in contact with tissue of rectum 318 adjacent prostate 320.

Balloon inflation mechanism 334 is conventionally known and includes an inflation lumen 354 which is in fluid communication with an interior of balloon 332. Balloon inflation mechanism 334 supplies pressurized fluid or air through inflation lumen 354 into the interior of balloon 332 to inflate balloon 332 to a desired size and pressure.

Temperature sensing device 336 preferably comprises an elongate strip of a plurality of temperature sensors which are supported along an exterior surface of balloon 332. Temperature sensing device 36 extends between first end 348 and second end 350 of balloon 332 and senses temperature of tissue of rectum 318 proximate prostate 320. Temperature sensing device 336 is electrically connected to rectal thermometry unit 38 by sensing device connector assembly 337 so as to transmit signals correlating to sensed temperatures to rectal thermometry unit 338.

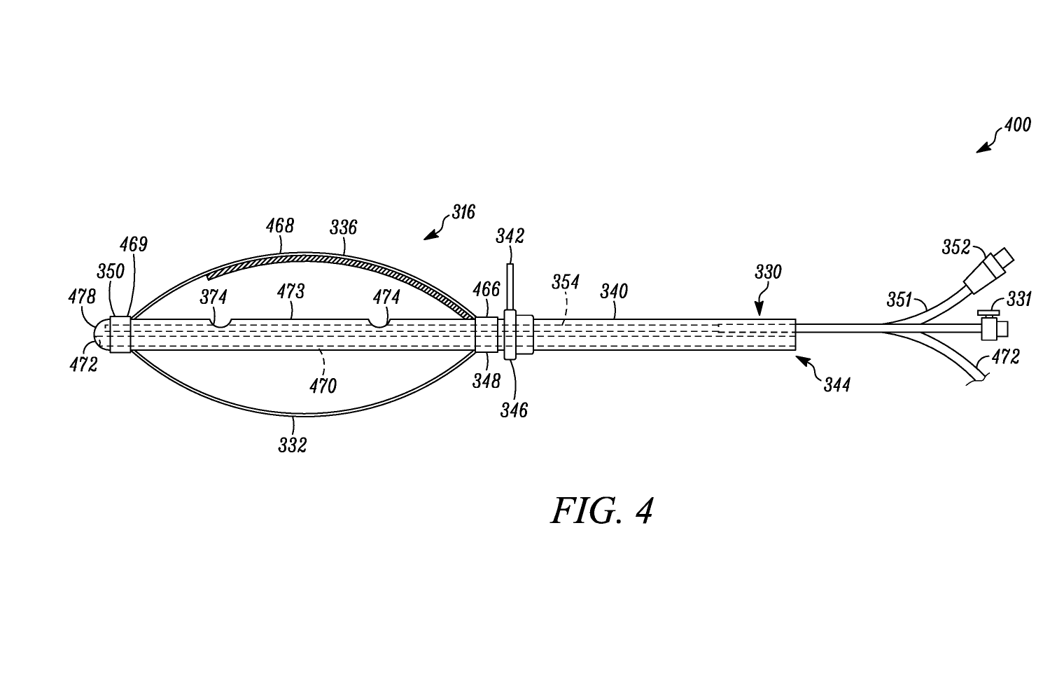

Sensing device connector assembly 337 connects temperature sensing device 336 and rectal thermometry unit 338 and includes cable 351 and connector 352. An electrode stimulation connector 472 can be separately positioned or integrated with other connectors. The electrode connector 472 (see FIG. 4) is in electrical communication with electrodes 394 and one or more of a power source, data storage and sensors (such as neural stimulation unit 341). Cable 351 preferably extends through central body 340 of handle 330 and has a first end connected to temperature sensing device 336 and a second end electrically connected to connector 352. Connector 352 preferably comprises a standard eight pin connector configured for mating with a corresponding connector of rectal thermometry unit 338.

Rectal thermometry unit 338 is conventionally known and includes cable 356 and connector 357. Connector 357 mates with connector 352 to electrically connect temperature sensing device 336 to rectal thermometry unit 338 for the transmission of electrical signals corresponding to sensed temperature values. Rectal thermometry unit 338 receives signals from temperature sensing device 336 and converts the signals into temperature values of the tissue of rectum 318. In one preferred embodiment, the temperature values are displayed and/or transmitted to transurethral thermal therapy system 312 for closed loop temperature control of system 312.

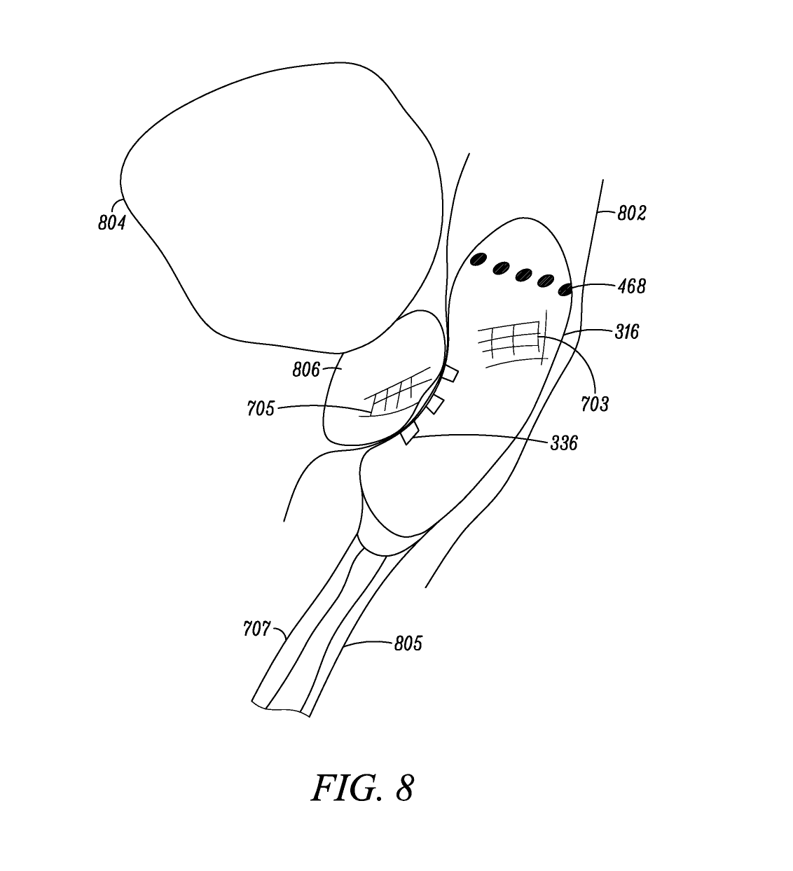

In FIG. 8, an alternate positioning of a conduction path from an RTU 316 and position of electrodes in proximity or contact with one or more of the sacral plexus 706, prostatic nerve plexus 705 and rectal nerve plexus 703 is shown. The conduction path can be a unipolar conduction path between RTU 316 and a surface electrode 468, such as with PTNS or could be bipolar between RTU 316 and a conductor on the urethral catheter or some combination of these. Sensing cables or connectors may electrically interface with the RTU and neural stimulation and sensing components, such as temperature sensor cable 707 and neural stimulation cable 705. Temperature sensors 336 can be positioned in proximity to the prostate and rectum, such as in contact with or integrated with the positioned RTU balloon. The electrodes 468 can be integrated with the RTU balloon 316 in proximity or in contact with one or more of the bladder and rectum, for example.

Stimulation from the cathode may be preferable for this type of therapy. Negative current from the cathode reduces voltage outside the neuronal cell membrane, causing depolarization and an action potential. The anode injects positive current outside making depolarization more difficult. Cathodal stimulation can significantly reduce (by as much as one third) the current that is required to elicit a neuronal response. Direct electrical current flowing through two electrodes on a given nerve will stimulate the nerve at the cathode (negative electrode) and resist excitation at the anode (positive electrode). The cathode is usually attached to the stimulating needle/catheter, the anode to the patient's skin as a returning electrode.

FIG. 4 shows a perspective view 400 of thermosensing unit 316 with the interiors of handle 330, balloon 332 and balloon supporting member 473 shown in dashed lines to illustrate the relationship between handle 330, balloon 332 and balloon supporting member 473. The one or more electrodes 394 of the present embodiments can be integrated on an outer surface of balloon 332. Further, support and placement mechanisms can be utilized to place and hold the electrodes in place, such as barbs, anchors, stent-like mesh, protrusions, or high adhesion materials, for example.

Balloon supporting member 473 may be composed of a material such as urethane or silicone, for example, and may be attached to handle 330 by adhesive or thermal bonding, for example, to form a two-piece elongate member along the length of the unit. In another embodiment, balloon supporting member 473 is an extension of handle 330 as a single elongate member spanning the unit. Central body 340 of handle 330 includes isolated lumens defining sensor cable lumen 462, balloon inflation lumen 354 and insertion lumen 470. Balloon inflation lumen 354 extends through body 340 of handle 330 from first end 344 to second end 346 of handle 330, and also extends through the interior of balloon 332 within balloon supporting member 473 to tip 478. Inflation openings 474 are provided in balloon supporting member 473 so that inflation lumen 354 is in fluid communication with the interior of balloon 332. Inflation lumen 354 is in fluid communication with control valve 331, and is preferably sized for transmitting a pressurized fluid from balloon inflation mechanism 34 (shown in FIG. 3) into the interior of balloon 332 through inflation openings 474 to inflate balloon 332.

Sensor cable lumen 462 extends substantially parallel to lumen 354 along the length of handle 330 from first end 344 to second end 346. Lumen 462 is sized for receiving sensor cable 351. Sensor cable 351 extends through lumen 462 to temperature sensing device 336 at second end 346 of handle 330.

Insertion lumen 470 extends substantially parallel to lumens 354 and 62 along the length of handle 330 from first end 344 to second end 346, and further extends through the interior of balloon 332 within balloon supporting member 473 to tip 478. Balloon 332 includes proximal waist 466 and distal waist 469, and is preferably integrally formed as part of a unitary structure of the same material. Balloon 332 has an outer diameter, when inflated, that radially increases from proximal balloon waist 466 to a point 468 generally midway between proximal balloon waist 66 and distal balloon waist 469, and that radially tapers from point 468 to distal balloon waist 469. Balloon 332 supports temperature sensing device 336 against the tissue of rectum 318 as shown in FIG. 3. Temperature sensing device 336 may be bonded to the inside or outside surface of balloon 332, or may be attached and contained in a groove formed in balloon 332 as described in further detail in U.S. Pat. No. 5,792,070 entitled RECTAL THERMOSENSING UNIT, by J. Kauphusman, J. Flachman and B. Neilson, which is hereby incorporated by reference. Distal waist 469 is located at a second end 350 of balloon 332 to allow fusing to balloon supporting member.

Urinary incontinence may be treated by stimulating the sacral nerves (see FIG. 7), which affect bladder control. Embodiments include stimulating from the rectum, urethra, posterior tibial nerve and combinations thereof. Such stimulation is effective in treating chronic pelvic pain, fecal incontinence, nocturnal urinary frequency, interstitial cystitis symptoms of urinary frequency, urinary urgency, and urinary urge incontinence, and overactive bladder symptoms of urinary frequency, urinary urgency and urinary urge incontinence. One such treatment may be done in a percutaneous manner by inserting a fine gauge needle into the posterior tibial nerve just above the ankle and applying an electrical stimulation to the needle. The tibial nerve carries the electrical stimulation up the leg to the S3 region of the lower spinal cord.

An example of an electrical nerve stimulator is disclosed in U.S. Pat. No. 6,493,588 entitled “Electro-nerve stimulator systems and methods,” which issued Dec. 10, 2002 to Malaney et al., and which is hereby incorporated herein in its entirety by reference thereto. The electrical leads for the stimulator disclosed in Malaney et al. are capable of being reused.

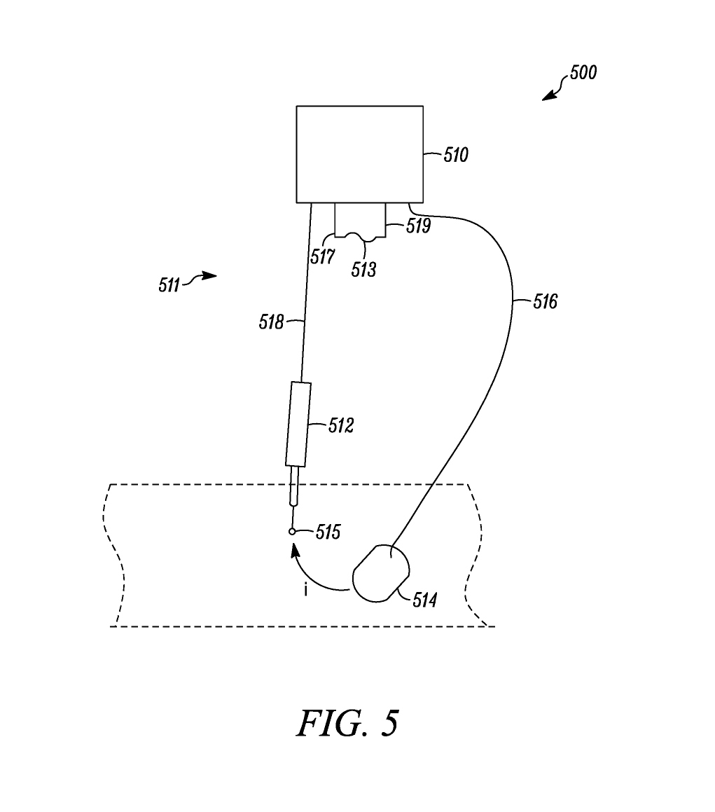

FIG. 5 shows 500 an exemplary nerve stimulator 511 that has a set of single-use electrical leads. The leads are shown in FIG. 5 as individual wires emerging from a controller 510, although it will be understood that any or all of the leads may be packaged in a connector to simplify connecting or removing the leads from the controller 510.

The controller 510 may include circuitry for monitoring time and generating a prescribed amount of current. The current may be AC, DC, or may alternatively be pulsed. The controller 510 may also include a battery or may alternatively use an external source of power.

As shown in FIG. 5, the controller 510 has four electrical connections or leads, although it may alternatively have more or fewer electrical connections, such as 3, 5, 6 and so forth. In the example of FIG. 5, two leads 516 and 518 provide a current through a portion of the patient's body, and the other two leads 517 and 519 are used for ensuring that the electrical leads are used for only a single therapy session.

Lead 516 connects the controller 510 to a transcutaneous electrode 514 that is placed in a suitable location on the exterior of the patient's body. The electrode 514 may be conductive or one or both sides, and may optionally have an adhesive that sticks to the patient's body.

Lead 518 connects the controller 510 to a percutaneous electrode needle 512 that is inserted into the patient's body at a stimulation site 15 and connects electrically near a nerve or bundle of nerves to be stimulated. The connector generates a voltage difference between leads 516 and 518, so that current flows between the electrode 514 and the needle 12. This current is denoted as arrow “i” in FIG. 5.

The remaining two leads 517 and 519 are connected to a fuse 513. The controller contains circuitry that can sense whether or not fuse 513 is blown, by generating a relatively small voltage between leads 517 and 519 and sensing whether or not any current is flowing through leads 17 and 19. The controller also contains circuitry that can deliberately blow the fuse 513, by generating a relatively large voltage between leads 517 and 519. The voltages used in leads 517 and 519 are determined in part by the fuse 513, and are typically low enough to not damage any tissue if it should come in contact with the leads 517 or 519.

Illustratively, the fuse 513 and the electrical leads 516, 517, 518 and 519 are bundled together at the controller 510 and connect to the controller 510 through a connector. A simulator 620 having an exemplary connector 622 is shown in FIG. 6, along with typical a controller unit 621, leads 625 and 626, an electrode 627, and a clip 628 that attaches to a needle 629. These elements are described in greater detail below. The descriptions in the following paragraphs are merely exemplary.

The controller unit 621 may be a portable unit, with a lightweight, ergonomic, handheld design. The controller unit 621 may have electronic touch pad controls or other suitable buttons or switches for entering data or changing the status of the controller unit 621. For instance, the controller unit 621 may have raised and embossed buttons, which provide tactile feedback to the user while protecting the controller unit 621 from moisture, contamination, and so forth. It will be readily understood by one of ordinary skill in the art that the controller may use any combination of buttons, switches, joysticks, levers, or any other suitable adjustment mechanism. The controller unit 621 may have a display such as an LCD screen for providing operational status and visual feedback information to the user.

The controller unit 621 includes a connection site to mate with a connector 622. The connection site may be a one-way fit connection site to ensure that the connector 622 for the leads cannot be plugged in backwards. The connector 622 may be keyed to the connection site in any desired manner, such as extended ribs, chamfers, spacing of the pins or sockets, and so forth.

The controller unit 621 contains the electronics suitable for providing a current for stimulating the desired nerve in the patient. Although specific current settings are described in the following paragraph, it will be understood that any suitable current scheme may used, such as direct current (“DC”), alternating current (“AC”) with a continuously varying current, AC with a pulsed current, or a combination of any of these. The specific current settings in the following three paragraphs are merely exemplary. Such parameters also apply to stimulation from the control unit 621 or other therapy device used for lower urinary tract or bowel therapy procedures.

The controller unit 621 may have twenty current setting levels, ranging from level “0” to level “19”, representing a current range of 0 mA to approximately 9 mA. At level “0”, the device produces 0 mA current. At level “1”, the current is 0.15 mA. At level “2”, the current level is 0.5 mA, and so forth. Each subsequent level represents a 0.5 mA increase. It will be understood that other increments and levels may be used as well.

The controller unit 621 may provide a pulsed current. The frequency of the pulses may be fixed at 20 Hz. The pulse width may be 200 microseconds. The pulse waveform may be square. It will be understood that other suitable frequencies, pulse widths, and waveforms may be used as well, and may optionally be adjustable.

The internal resistance of the controller unit 621 may be varied by the controller unit 621 from 500 to 4000 Ohms in order to provide the desired current. This range is merely exemplary, and other suitable ranges may be used as well.

The leads transfers the electrical current from the controller unit 621 to the tibial nerve and may include one or more lead sets such as lead set 623, one or more percutaneous electrodes such as needle electrode 629, one or more surface electrodes such as electrode 627, and an optional alcohol pad (not shown). The following description of these elements is merely exemplary.

The components of the lead set 623 create the non-sterile circuit interface between the controller unit 621 and the patient. A one-way fit connector 622 is attached to the proximal end of the lead wire 623. The distal end of the lead wire may be split into individual wires 625 and 626, with the split occurring at a fork 624. One wire 625 may be attached to an adhesive-backed surface electrode 627; the other wire 626 may be attached to a needle electrode clip 628.

The needle electrode clip 628 clips to the needle electrode 629. The needle electrode 629 illustratively is a 34 gauge solid stainless steel needle, which may be packaged within a plastic guide tube with stop plug. Each needle electrode 629 may be supplied sterile (ethylene oxide) in an individual peel-open package. The alcohol pad may be prepackaged to clean the needle electrode insertion site.

The following paragraphs describe a preferred set of instructions for use of the stimulator. These instructions are merely exemplary. Any suitable instructions may be used. Percutaneous tibial nerve stimulation (PTNS) therapy involves placing the needle electrode 629 into the lower, inner aspect of either leg slightly cephalad to the medial malleolus. The surface electrode 627 is placed over the medial aspect of the calcaneous on the same leg. The lead set 623 first is plugged into the stimulator 621, and then the needle electrode clip 628 is clipped to the needle electrode 629. The stimulator 621 produces an adjustable electrical pulse that travels to the sacral nerve plexus via the tibial nerve. Among other functions, the sacral nerve plexus regulates bladder and pelvic floor function.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. All documents referred to herein are hereby incorporated by reference for any purpose. However, if any such document conflicts with the present application, the present application controls. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A method of reducing patient discomfort due to a procedure of the lower urinary tract, prostate or bowel, the method comprising:
electrically contacting one or more afferent or efferent nerve fibers that innervate the patient's prostate, bladder, or both, wherein the electrical contact occurs during the time a minimally invasive or surgical procedure of the lower urinary tract, prostate, or bowel is performed on said patient, and wherein the electrical contact is sufficient to reduce patient discomfort during and optionally after the procedure.

2. The method of claim 1, wherein electrically contacting comprises one or more of blocking a neural signal and stimulating one or more nerves.

3. The method of claim 1, wherein the discomfort comprises one or more of pain, urgency and bladder spasms.

4. The method of claim 1, wherein the procedure comprises one or more of transurethral microwave thermotherapy of the prostate, transurethral needle ablation of the prostate, microwave therapy to treat bladder cancer, cystoscopy, colon polyp removal, prostate biopsies, transurethral energy based procedure to treat prostrate and invasive urodynamics.

5. The method of claim 1, wherein the one or more afferent or efferent nerve fibers comprise nerve fibers in or near one or more of the rectum, bladder, perineum, urethra, and leg of the patient.

6. The method of claim 1, wherein the one or more electrodes are integrated with a thermal sensing unit.

7. The method of claim 1, wherein electrically stimulating produces sinusoidal waveforms, biphasic waveforms, or both, said waveforms having frequencies of about 1 kHz to about 100 kHz.

8. The method of claim 7, wherein a current of the waveforms ranges from about 0.5 mA to about 15 mA.

9. A method of stimulating nerves during a medical procedure comprising:
positioning one or more electrodes in electrical contact with one or more afferent or efferent nerves in a patient before or during a minimally invasive or surgical procedure of the lower urinary tract, prostate, or bowel; and
during said procedure, electrically stimulating one or more afferent or efferent nerves as an ancillary procedure sufficient to affect one or more of bladder activity, prostate function, incontinence, and pain in said patient.

10. The method of claim 9, further comprising positioning one or more electrodes in electrical contact with one or more nerves in or near one or more of the rectum, bladder, perineum, urethra, and leg of the patient.

11. The method of claim 10, further comprising electrically stimulating the one or more nerves in or near one or more of the bladder, perineum, urethra, and leg simultaneously with electrically stimulating one or more nerves from within the rectum.

12. The method of claim 10, further comprising electrically stimulating the one or more nerves in one or more of the bladder, perineum, urethra and leg sequentially with electrically stimulating one or more nerves from within the rectum.

13. The method of claim 9, wherein the procedure comprises one or more of transurethral needle ablation of the prostate, transurethral microwave thermotherapy of the prostate, prostate biopsy, microwave therapy to treat bladder cancer, transurethral energy based procedure, colon polyp removal, invasive urodynamics and cystoscopy.

14. The method of claim 9, wherein the one or more electrodes comprises an array of electrodes.

15. The method of claim 9, wherein the one or more electrodes are integrated with a thermal sensing unit.

16. The method of claim 9, wherein electrically stimulating produces sinusoidal waveforms, biphasic waveforms, or both, said waveforms having frequencies of about 1 kHz to about 100 kHz.

17. The method of claim 16, wherein a current of the waveforms ranges from about 0.5 mA to about 15 mA.

18. A lower urinary tract, prostate, or bowel neuromodulation system, the system comprising:
one or more electrodes, at least one of the electrodes positioned in contact with one or more afferent or efferent nerves in proximity to the urinary tract, prostate, or bowel of a patient;
one or more temperature sensors;
an electrical stimulation module, in electrical communication with the one or more electrodes; and
a controller, in electrical communication with one or more of the one or more electrodes, one or more temperature sensors, a microwave generator, and an electrical stimulation module;
wherein the one or more electrodes are capable of stimulating one or more afferent or efferent nerves as an ancillary procedure during a minimally invasive or surgical procedure of the lower urinary tract, prostate, or bowel sufficient to affect one or more of bladder activity, prostate function, incontinence, and pain in said patient.

19. The neuromodulation system of claim 18, wherein at least one of the one or more electrodes are positioned in electrical contact with one or more nerves in or near one or more of the rectum, bladder, perineum, urethra, and leg of the patient.

20. The neuromodulation system of claim 18, wherein the one or more temperature sensors comprise rectal temperature sensors and microwave delivery system temperature sensors.
